(19) United States
(12) Plant Patent Application Publication
(10) Pub. No.: US 2006/0225169 P1
(43) Pub. Date: Oct. 5, 2006

(54) Koroneiki IRTA I-38 olive tree (Olea europaea)

(50) OLEA EUROPAEA; OLIVE

(76) Inventor: Joan Tons, Subirats (ES)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 11/093,775

(22) Filed: Mar. 29, 2005

(51) Int. Cl. A01H 5/00

(52) U.S. Cl. PLT/158

(57) ABSTRACT

This new invention of an olive plant is characterized by its similarity to the ‘Koroneiki Standard’ (unpatented) from the island of Crete (Greece) from which it is a clonal selection, but is distinguished therefrom by producing a very small fruit early in the age of the tree (3rd leaf), better vigor, and higher productivity. The plants are ideally suited for olive oil production, mechanical harvesting and high density planting. The fruit matures in northern California during the third week of October.

### OLIVE TREE

(Olea europaea)

### BACKGROUND OF THE NEW VARIETY

The present invention relates to a new and distinct variety of olive plant, Olea europaea, and will hereafter be denominated varietally as ‘Koroneiki IRTA I-38’ and more particularly to a olive plant that produces fruit for harvest during the third week of October in the Gridley area of the northern Sacramento Valley in California and which is further distinguished as to novelty by having better vigor and fruit production than ‘Koroneiki Standard’ (non-patented) variety. The variety ‘Koroneiki Standard’ has been used in Greece (Crete) for olive oil production over the past decade.

### ORIGIN AND ASEXUAL REPRODUCTION OF THE NEW VARIETY

This new variety was vegetatively reproduced by cuttings after clonal section in Greece, and shipped and planted at two sites in Spain in 1995: Cataluna and Andelucia. After several years of evaluations, these clonal selections, chosen for the characteristics of medium vigor and very good production, were vegetatively propagated in 1997 and shipped to the United States. After completion of post-entry quarantine requirements in Oregon, in 2001 the vegetatively propagated material was shipped to Gridley, Calif. In 2004, 60,000 cuttings were asexually propagated.

### SUMMARY OF THE NEW VARIETY

The new variety of olive tree is characterized by having the general characteristics or the ‘Koroneiki Standard’ variety but having the important distinction of having better vigor and very good production, maturing in northern California in the third week of October. Since this olive tree is smaller than the standard varieties (e.g. ‘Manzanillo.’), the new variety is well suited for mechanical harvesting and high density planting (i.e. 670 plants/acre).

### BRIEF DESCRIPTION OF THE DRAWINGS

FIG. 1 is a color photograph exhibiting the overall shape of the tree and its appearance at third leaf.

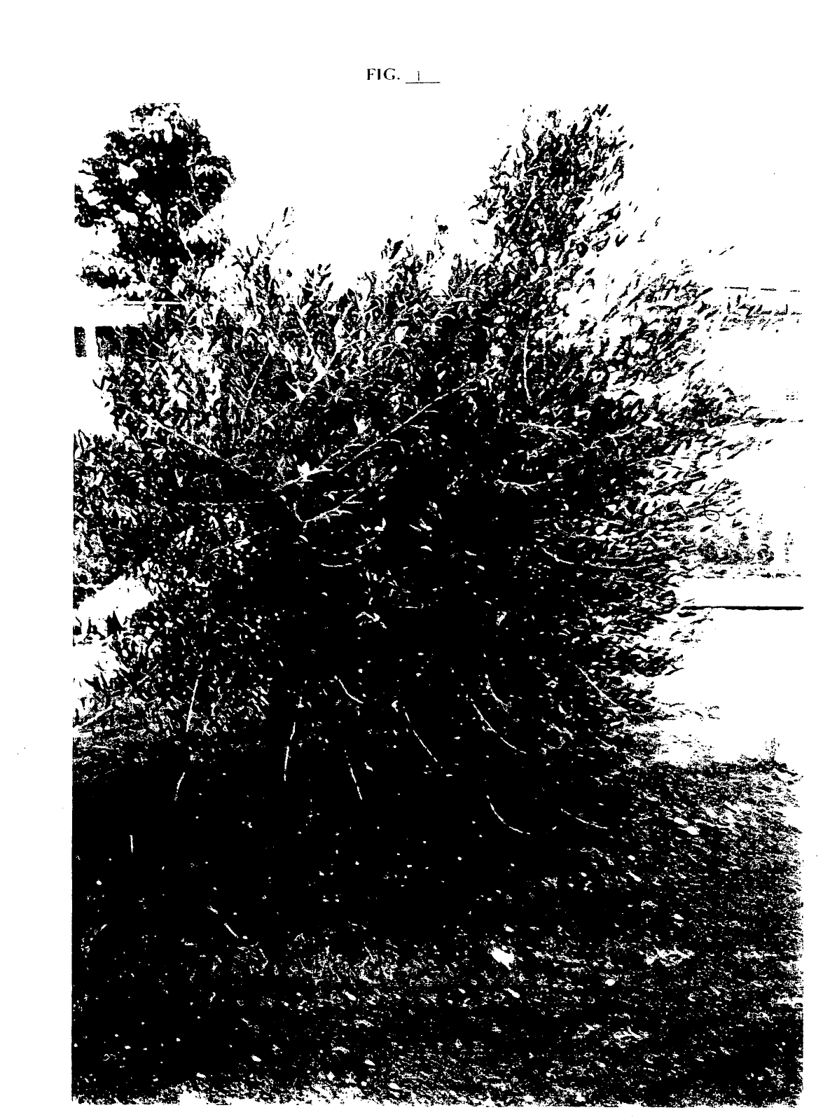

FIG. 2 is a color photograph of a typical branch of the new variety bearing fruit.

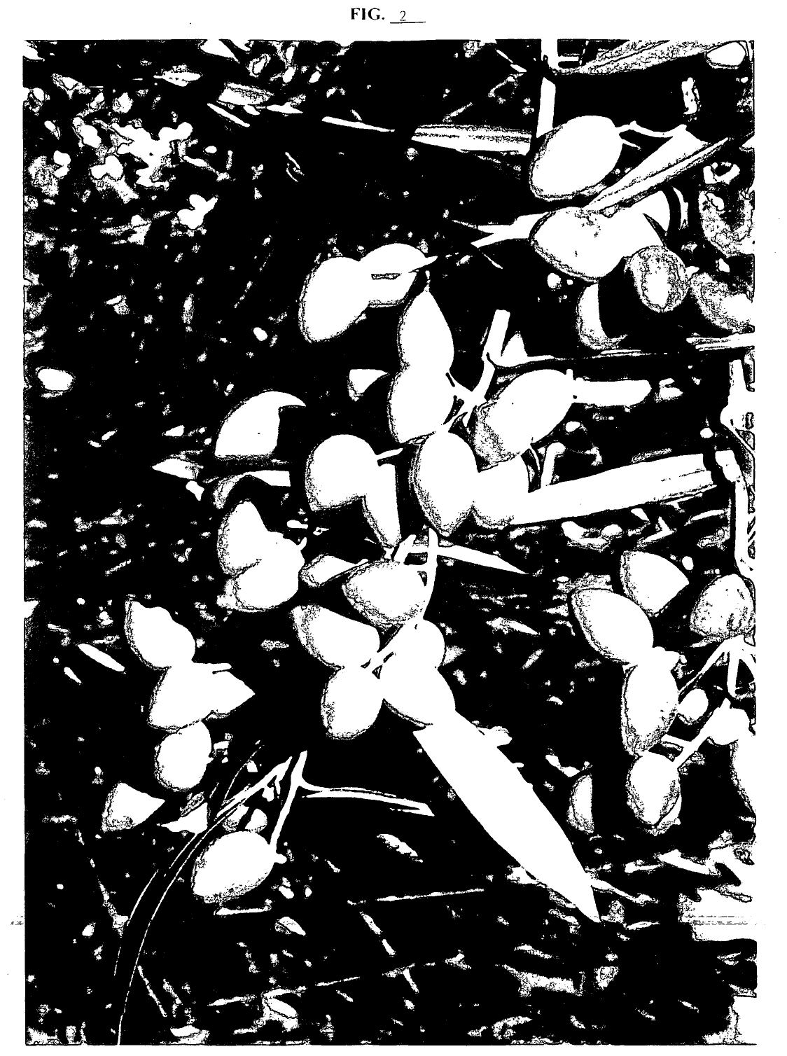

FIG. 3 is a color photograph of another typical branch of the new variety bearing fruit.

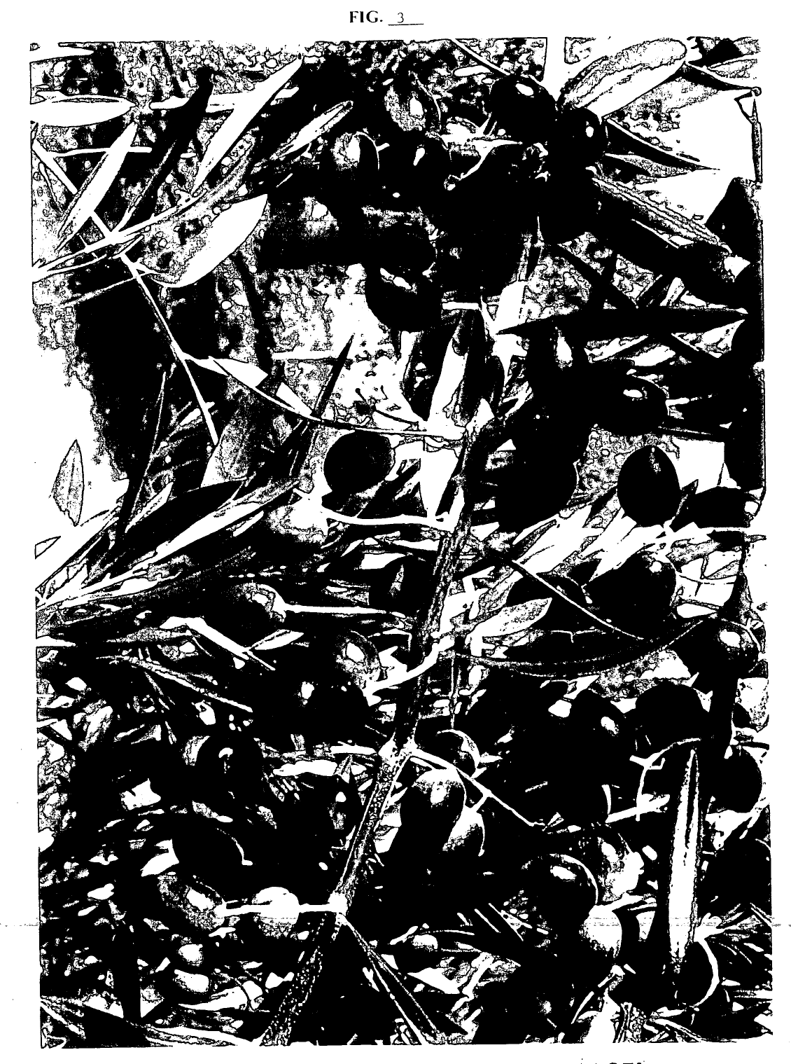

FIG. 4 is a color photograph showing the foliage in the upper portion, a green fruit on the left in the middle, a seed in the middle, and a ripe fruit on the right in the middle; at the bottom of the photograph is a fruit cut in half displaying the seed in one half on the left and the seed cavity and flesh at the seed cavity on the right.

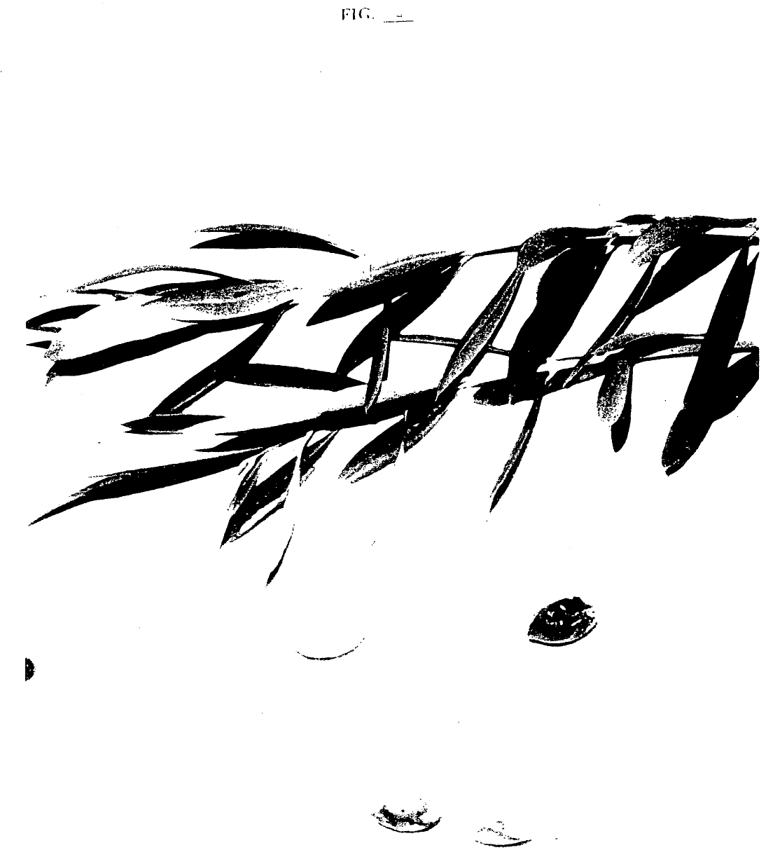

### DETAILED DESCRIPTION

Referring more specifically to the horticultural details of the new and distinct variety of olive trees, the following descriptions have been observed under the ecological details prevailing at the site of origin which is located near Gridley, Calif. in the Sacramento Valley of northern California. All major color designations are by reference to the Dictionary of color by Maerz & Paul, First Edition, 1930. Common colors are also employed.

### Tree

Height: small, 6-8 feet (183-244 cm).
Width: 2.5-3.5 feet (76-107 cm).
Trunk:
   Circumference.—7.5 inches (19 cm), 12 inches (30 cm) above ground.
   Bark.—smooth.
   Color of bark.—Dove grey (45-B-1). p0 Vigor: medium good.
   Chilling.—normal for olive trees in Northern California.
Figure: open.
   Lenticels.—small.
Branches: 4.5 inches (11 cm) in circumference, 16 inches (41 cm) above crotch.
   Color.—Mature: Rainette Green. (21-K-4) Immature: Certosa (20-L-3).
   Lenticels.—very small, moderate number.

### Leaves

Size: small.
Average Length: 45.5 mm (1.77 inch).
Average Width: 10 mm (0.39 inch).
Color:
   Upper surface.—Mt. Vernon Green (23-J-8).
   Lower surface.—Fern (21-I-7).
Shape: elliptical.
Marginal Form: entire (complete).
Midvein:
   Color.—Fern (21-I-7).
   Thickness.—Less than 1 mm (0.04 inch).
Petiole:
   Size.—short, 4.3 mm (0.17 inch) in length.
   Color.—Certosa (20-L-3).
Stipules:
   Size.—small, 1 mm (0.04 inch).
   Color.—Certosa (20-L-3).

### Fruit

Fruit: Borne in clusters of 2-3.
   Size.—Small. Length: 18-19 mm (0.70-0.74 inch). Width: 12-13 mm (0.47-0.51 inch).
Apex: pointed.
Base: truncate.
Ground Color: Grey Drab, Quaker Gray (21-B-1) ranging to Kara Dagh (56-A-2).
Flesh Color: Grey Drab (21-B-1).
   Cavity.—Length: 10 mm (0.39 inch). Width: 4 mm (0.16 inch).
   Color of cavity.—Olive Grey, Scotch Grey (21-A-2).
Seed:
   Small.—cling type.
   Length.—13-14 mm (0.51-0.55 inch).
   Width.—7 mm (0.27 inch).
   Shape.—elliptical and asymmetrical in shape.
   Base.—truncate.
   Sides.—slightly asymmetrical unequal.
   Apex.—pointed.
   Ridges.—uneven.
   Suture.—marked.
   Tendency to split.—none known.
Use: olive oil production.
   Keeping and shipping quality.—good.
   Resistance to disease.—Verticilium and Peacock Spot.

Harvesting.—second week of October.

CHEMICAL ANALYSIS
Olive Oil - Koroneiki IRTA I-38

| | Percentage of Total |
|---|---|
| Type of Fatty Acid | Oil Content |
| C16.0 Palmitic Acid | 11.4 ± .49 |
| C16.1 Palmitoleic Acid | 8.40 ± .18 |
| C18.0 Estaric Acid | 2.51 ± .29 |
| C18.1 Oleicv Acid | 76.6 ± .59 |
| C18.2 Linoleic Acid | 6.89 ± .74 |
| C18.3 Linolenic Acid | 0.93 ± .29 |

M/P ratio (Monosaturated Fat/Polysaturated Fat Ratio): 10.0 ± 1.02
Polyphenols (PPM Catecol): 600 ± 167
Bitterness 0.45 ± 09

What is claimed is:

1. A new distinct olive plant as described and illustrated that is characterized by having the general characteristics of the ‘Koroneiki Standard’ (unpatented) variety but is distinguished therefrom in that it has better vigor and higher productivity, the plants being ideally suited for olive oil production, mechanical harvesting and high density planting, with the fruit maturing in northern California in about the third week of October.
